(19) United States
(12) Patent Application Publication
(10) Pub. No.: US 2007/0048338 A1
(43) Pub. Date: Mar. 1, 2007

(54) Compositions and methods for surface treatment in medical and surgical procedures

(76) Inventor: Byron S. Ladd, Midlothian, VA (US)

Correspondence Address: TROUTMAN SANDERS LLP, 600 PEACHTREE STREET , NE, ATLANTA, GA, 30308, US

(21) Appl. No.: 11/510,172

(22) Filed: Aug. 25, 2006

Related U.S. Application Data
(60) Provisional application No. 60/711,929, filed on Aug. 26, 2005.

(51) Int. Cl. A61K 31/56; A61K 31/24; A61K 33/36; A61K 9/00

(52) U.S. Cl. 424/400; 424/672; 514/171; 514/537

(57) ABSTRACT

The present invention comprises compositions and methods for contemporaneously anesthetizing and antiseptically treating or pretreating anatomic surfaces for invasive surgical or treatment procedures. In particular, compositions and methods according to the present invention are used to treat ocular surfaces for intravitreal injections and other ophthalmic procedures. Compositions of the present invention comprise antiseptic agents and anesthetic agents in an aqueous gel or semi-gel formulation base to provide for enhanced methods of treating anatomic surfaces such as the eye prior to surgical or other invasive procedures.

### RELATED APPLICATIONS

This application claims the benefit of U.S. Provisional Patent Application No. 60/711,929, filed Aug. 26, 2005, which is herein incorporated by reference in its entirety.

### TECHNICAL FIELD

This invention is related to compositions and methods for their use for treatment and pretreatment for procedures for medicine, surgery, and dentistry. In particular, the compositions and methods according to the present invention provide for the one-step preparation of a suitable antiseptic field in addition to achieving anesthesia for an anatomic surface as a treatment or as a pretreatment for medical or surgical procedures.

### BACKGROUND OF THE INVENTION

Medical and surgical procedures often involve treating an anatomic surface to achieve antiseptic and anesthetic qualities, especially as a pre-treatment for an invasive procedure on or through that anatomic surface. To achieve appropriate antisepsis and anesthesia, antiseptic and anesthetic agents must be administered in forms that are biologically and chemically both tolerated by the anatomic surface and capable of sufficient absorption by or through the anatomic surface to achieve the desired antiseptic and anesthetic qualities within the anatomic surface and/or its underlying tissues.

The field of ophthalmology is one area of medicine and surgery that often requires treating an anatomic surface (such as the ocular surface, cornea and ocular adnexa) as part of a procedure. Over the past several years, there has been a marked increase in the number of intravitreal injections (IVI) performed for the treatment of retinal pathology. IVI entails inserting a needle through the sclera into the posterior segment of the eye. Previously, intravitreal injections were utilized for the delivery of antibiotics in the management of intraocular infections, which included post-operative and endogenous endophthalmitis, and Cytomegalovirus retinitis. Intravitreal injections of gas during a pneumatic retinopexy are also utilized for the management of retinal detachments. Due to the low incidence of these indications, IVI was once an uncommon procedure.

However, in contemporary retinal practice, IVI is a commonly performed procedure. This change has resulted from the rapid adoption of intravitreal steroids for the treatment of diabetic retinopathy, retinal vascular occlusions, and wet, age-related macular degeneration (AMD). Due to the high prevalence of these diseases, the number of IVI's has grown exponentially. In addition, pegaptanib (Macugen™) and ranibizumab (Lucentis™) have been approved by the U.S. F.D.A. for the treatment of wet, age-related macular degeneration. Pegaptanib therapy requires intravitreal injections every 6 weeks for a year. In the United States, approximately 300,000 individuals will develop wet AMD per year. Thus, a potential 2.7 million injections per year could be necessary to manage AMD alone. Furthermore, the incidence of AMD is expected to triple over the next 20 years as the U.S. population ages.

As performed using conventional materials and methods, anesthesia for IVI currently entails using a topical anesthetic prior to sterile antiseptic surface preparation followed by additional topical proparacaine on a cotton swab and a 0.1 cc injection of subconjunctival 1% lidocaine at the intended IVI site. Conventional practice involves separate steps for anesthesia and antiseptic surface preparation. Theoretically, these steps add additional risks for re-introducing bacteria to the injection site in many ways. First of all, the additional anesthesia is given after the iodine antiseptic preparation. These simple steps also add multiple supplies to the procedure, with each item potentially increasing the risk of contamination. Additional items include a filtered and non-filtered needle, syringe, cotton swab, bottle of topical proparacaine, and a vial of lidocaine. The technical maneuvers of a subconjuctival injection can often require awkward positioning and risks needle contact with potentially unsterile lids and lashes, which may then contaminate the subconjuctival space.

IVI is not without potential complications. These include intravitreal hemorrhage, retinal detachment, cataracts, and endophthalmitis. Most intraocular procedures have a risk of endophthalmitis around 1/5,000. However, for unclear reasons the rate of endophthalmitis secondary to IVI has been reported much higher, from 0.03% to 0.8%. To date, the only proven steps to reduce the rate of endophthalmitis are using a lid speculum and washing the eye pre-operatively with a 5% Povidone-Iodine solution. Improvements in ocular preparation and WI technique could result in a safer procedure.

Conventional procedures employing separate aqueous antiseptic and anesthetic agent applications are limited in their effectiveness as most of the liquid is lost due to run-off due to the contour of the eye and eyelids, thereby limiting the beneficial amount of contact time between agents and the ocular surfaces. The use of a gel-based composition including both antiseptic and anesthetic agents would offer significant advantages over conventional materials and methods by increasing contact time and efficiency between the antiseptic and anesthetic agents and the ocular surface. Moreover, the inclusion of antiseptic and anesthetic agents in a gel-based ophthalmic composition would allow surface antiseptic preparation and anesthesia to be achieved in a one-step procedure.

### SUMMARY

The present invention comprises compositions and methods for treating an anatomic surface of a body in a one-step procedure to achieve antisepsis and anesthesia thereon. The compositions of the present invention comprise antiseptic agents combined with anesthetic agents in a viscous gel or semi-gel formulation base that, in use, remains in contact with the anatomic surface so that the selected area is rendered sufficiently antiseptic and anesthetized for treatment or as pretreatment for invasive procedures. An embodiment of the composition comprises povidone-iodine combined with lidocaine, in an aqueous hydroxymethylcellulose gel or semi-gel formulation base. An aspect of the present invention comprises compositions and methods for intravitreal injections.

Methods of the present invention comprise methods for making the compositions of antiseptic agents combined with anesthetic agents in an aqueous gel or semi-gel formulation base and methods of using the compositions for medical or surgical treatment or pretreatment. Methods for making the compositions may comprise mixing the component antiseptic agents, anesthetic agents, and gel or semi-gel formulation base together in effective amounts to provide a composition that is sufficiently viscous that the composition is retained on the desired anatomic surface but does not tightly adhere to that surface so that it may easily be removed by irrigation or other simple mechanical cleansing procedures. Procedures in which methods using the compositions of the present invention may be employed include, but are not limited to, ophthalmic procedures like intravitreal injections, anterior chamber paracentesis, retinal cryopexy, cataract surgery, iridotomy, trabeculotomy, trabeculoplasty, glaucoma surgery, ophthalmic implant surgery, and pars plana vitrectomy.

Other fields of medicine may utilize the compositions and methods of the present invention to achieve antiseptic and anesthetic effects in a one-step procedure in situations such as, but not limited to, the repair of cutaneous lacerations, procedures or treatments mucous membrane-lined surfaces, dental procedures and treatments, and myringotomies.

### DETAILED DESCRIPTION

The present invention may be understood more readily by reference to the following detailed description of the compositions and methods of the invention and the examples included herein. However, before the compositions and methods of the present invention are disclosed and described, it is to be understood that this invention is not limited to the exemplary embodiments described within this disclosure, and the numerous modifications and variations therein that will be apparent to those skilled in the art remain within the scope of the invention disclosed herein. It is also to be understood that the terminology used herein is for the purpose of describing specific embodiments only and is not intended to be limiting.

Unless otherwise noted, the terms used herein are to be understood according to conventional usage by those of ordinary skill in the relevant art. In addition to the definitions of terms provided below, it is to be understood that as used in the specification and in the claims, “a” or “an” can mean one or more, depending upon the context in which it is used.

As used herein, the terms “antiseptic agents” and “antiseptics,” which terms may be used interchangeably herein, are substances which may be used to reduce microbial levels and are biologically compatible enough to be applied to a particular anatomic surface without causing substantial irritation, inflammation, dysfunctional or other undesired reactions on or within the anatomic surface or adjacent tissues or organs. Antiseptic agents used in compositions according to the present invention may be microbicidal (bacteriocidal, fungicidal, and/or viricidal) in their actions, and are intended to provide a reduction in the ambient flora in the anatomic surface onto which they are administered.

As further used herein, the terms “anesthetic agents” and “anesthetics,” which terms may be used interchangeably herein, are substances which may be used to induce anesthesia, or reversibly depress neuronal function, producing total or partial loss of pain sensation when administered to an anatomic surface or tissue.

As further used herein, the terms “gel or semi-gel formulation base,” “gel,” and “semi-gel,” which terms may be used interchangeably herein, are viscous aqueous substances which may be used to deliver antiseptic agents and anesthetic agents to an anatomic surface in compositions and methods according to the present invention.

The present invention comprises compositions and methods for medical and surgical procedures or treatments involving an anatomic surface of a body. An anatomic surface of a body may include an ocular surface, a mucous membrane surface, a dermal surface, a visceral surface, or any combination thereof on or within a mammalian body. The compositions of the present invention comprise combinations of antiseptic agents with anesthetic agents in an aqueous gel or semi-gel formulation base that is desirably retained on an anatomic surface but may also be removed when desired, for example by wiping or washing with irrigation or other fluids. The present invention further comprises methods of using compositions of the present invention for medical or surgical treatment or pretreatment to achieve antisepsis and anesthesia on one or more desired anatomic surfaces in or on a body. Such methods may comprise a one-step procedure.

Methods for making compositions of the present invention may comprise admixing one or more antiseptic agents with one or more anesthetic agents and an aqueous gel or semi-gel formulation base, which may also include other components, such as antibiotics, other medicaments, diluents and buffers. In various embodiments according to the present invention, the composition may be provided in one sterile container or may be provided in sterile containers to be combined prior to use. The formulation of such gel and semi-gel formulation bases is well known to those skilled in the art.

Ophthalmic formulations of the invention are improvements over existing oil based formulations and solutions and from the sequential use of individual components. Though not wishing to be bound by any particular theory, it is believed that, upon administration, aqueous eye fluids mix with the gel or semi-gel composition of the present invention, resulting in release of the antiseptic agents and anesthetic agents. Also, a predetermined dose reaches the site being treated. Furthermore, a much higher percentage of the dose of the antiseptic agents and anesthetic agents is maintained in the eye than with conventional ophthalmic ointments or solutions.

The compositions of the present invention also allow desired amounts of pharmacologically active antiseptic and anesthetic agents to be applied such that these agents may slowly spread over the external anatomic surface of the eye. Furthermore, a one-step application of a composition according to the present invention provides more efficient delivery than the individual administration of the component agents contained therein.

The formulation of the invention provides a safe means for time release of antiseptic agents and anesthetic agents into the eye. The release rate depends on the viscosity of the gel or semi-gel formulation base, i.e., higher viscosity results in slower release. The invention relates in particular to gel and semi-gel formulation bases having relatively low viscosity and correspondingly more rapid release profile. In one embodiment of the present invention, the gel or semi-gel formulation base has a viscosity in the range of about 10,000 cps to about 50,000 cps. at about 25° C. based on Brookfield (LV) analysis, from about 10,000 cps to about 40,000 cps, 10,000 to about 30,000 cps, 20,000 cps to about 50,000 cps, from about 20,000 cps to about 40,0000 cps, from about 30,000 cps to about 50,000 cps, from about 30,000 cps to about 40,000 cps, or from about 40,000 cps to about 50,000 cps, and all ranges therein between.

Unlike gel ophthalmic applications disclosed by the prior art, the present invention is directed to use prior to an invasive ophthalmic procedure. Onset of pharmacological activity is therefore more desirable in use of compositions according to the present invention than enduring or delayed time-release qualities.

The formulation of compositions according to the present invention may be placed in any desired dispensing container or delivery device suitable for an ophthalmic formulation. The dispensing container or delivery device may be an ophthalmic delivery system, such as a sterile ophthalmic tube, e.g., a conventional 3.5 g tube having an ophthalmic tip and containing the ophthalmic formulation of the invention, or a sterile single use container containing 0.01-10.0 g or more of the formulation.

In various other embodiments of the present invention, the dispensing container or delivery device may be a pre-loaded syringe, a bottle, a vial, or other container or delivery device. In yet other embodiments according to the present invention the anesthetic agent and the antiseptic agent may be provided to the consumer in separate or multi-chambered containers or delivery devices for mixture at the time of use.

An embodiment of a composition of the present invention comprises an effective amount of an antiseptic agent in the form of an antiseptic, including but not limited to, povidone-iodine, benzalkonium chloride, and chlorobutanol, and an effective amount of an anesthetic agent, including but not limited to, lidocaine, tetracaine, proparacaine, and bupivacaine. These two are admixed in a viscous aqueous gel or semi-gel formulation base such as, but not limited to, hydroxymethylcellulose. An exemplary preferred composition of the present invention comprises povidone-iodine (about 50 mg/ml), lidocaine (about 20 mg/ml); and hydroxymethylcellulose (about 20 mg/ml), dissolved in about 1.0 ml of purified water. This example yields an ophthalmic gel comprising about 5% povidone-iodine, about 2% lidocaine, and about 2% methylcellulose. Other percentages and dose ratios are contemplated by the present invention, including povidone-iodine in an effective concentration range of about 0.01% to about 20%; lidocaine in a concentration range of 0.01% to about 35%; and methylcellulose in a concentration range of about 0.1 wt. % to about 15 wt. %.

Compositions of the present invention may comprise povidone-iodine as an antiseptic agent in a effective concentration of about 0.5%, about 1%, about 2.5%, about 5%, about 7.5%, about 10%, about 12.5%, about 15%, about 17.5%, and about 20%, where “about” means+/−1.25%.

Compositions of the present invention may comprise lidocaine as an anesthetic agent in a concentration of about 0.5%, about 1%, about 2.5%, about 5%, about 7.5%, about 10%, about 12.5%, about 15%, about 17.5%, about 20%, about 22.5%, about 25%, about 27.5%, about 30%, about 32.5% and about 35%, where “about” means+/−1.25%.

Compositions of the present invention may comprise methylcellulose as an aqueous gel or semi-gel formulation base in a concentration of about 0.05%, about 1%, about 2.5%, about 5%, about 7.5%, about 10%, about 12.5%, and about 15%, where “about” means+/−1.25%.

In compositions of the present invention one or more antibiotics may also be added to a gel antiseptic/anesthetic composition to provide additional bacteriostatic, fungistatic, and/or viristatic effects. Such antibiotics may include, but are not limited to: polymyxin B sulfate (from about 1,000- to about 100,000 units/gm) neomycin sulfate (from about 0.5- to about 25 mg/gm); gramicidin (from about 0.01- to about 5.0 mg/gm), zinc bacitracin (from about 100- to about 5000 units/gm), gentamicin (from about 0.01- to about 5%); chloramphenicol (from about 0.01- to about 5%);), tobramycin (from about 0.01-5 to about %);), erythromycin, (from about 0.5- to about 25 mg/gm), and tetracycline HCl (from about 0.01- to about 25%).

Gels or semi-gels in compositions of the present invention may comprise cellulose and its derivatives. Aqueous cross-linked acrylic polymers, poly acrylic acid, pluronic polyol polymers, other polynols, carboxy vinyl polymers, other carbomers, or other biologically inert materials that will not cause irritation to the corneal or other ophthalmic structure may also be employed in compositions of the present invention. Gels or semi-gels in compositions of the present invention may comprise dextran, polyvinyl alcohol, poyvinylacyrlates, and polymeric mixtures thereof.

Gels or semi-gels in compositions of the present invention may comprise methyl cellulose or carboxymethyl cellulose salt. Gels or semi-gels in compositions of the present invention may comprise any substance derived from cellulose that forms an aqueous gel or semi-gel at a desired viscosity, i.e., is soluble in water and forms a gel or semi-gel, may be used. Such cellulose derivatives are well known, as are their properties, and are described, e.g., in the U.S. Pharmacopeia 2005 (United States Pharmacopeial Convention, Inc., The United States Pharmacopeia/The National Formulary). Such cellulose derivatives include, but are not limited to, methyl cellulose, hydroxypropyl cellulose, methyl hydroxypropyl cellulose, hydroxypropyl methyl cellulose, cellulose acetate, ethyl cellulose, methyl hydroxyethyl cellulose, hydroxyethyl cellulose, and cellulose gum.

The gels of compositions of the present invention may further comprise one or more inorganic salts or salts of organic amines or amino acids in an amount effective to provide a gel or semi-gel with the desired viscosity. Sodium acetate is an exemplary salt that may be used for this purpose. Those skilled in the art will be capable of determining the appropriate quantity of such a salt to be added to a gel composition of the present invention. By way of example, however, sodium acetate concentrations in the range of about 0.01 to about 0.5% by weight of the gel have generally been found to be appropriate for providing gels of a suitable original and residual viscosity.

Gels for use on the eye may be isotonic. Ophthalmic gel compositions of the present invention may be isotonized by the use of suitable nonionic agents. Commonly, sorbitol or mannitol may be used for this purpose. Glycerol may also be used. The eye tolerates osmolarities in the range of 100-450 mOsmol/L

Low to medium viscosity cellulose based agents may be used in compositions of the present invention. Such agents have a lower number of substituents, such as methoxy-, ethoxy-, hydroxy- propyl- and carboxy-substituents, attached to the cellulose backbone than high viscosity cellulose based agents. Some gels or semi-gels in compositions of the present invention may comprise dextran, polyvinyl alcohol, poyvinylacyrlates, and polymeric mixtures thereof, however, a higher concentration may be used, constituting from about 5.0 to about 15 wt. % or more of the formulation.

Suitable gels or semi-gels for compositions of the present invention are commercially available. A composition according to the present invention may contain additional pharmaceutically inactive substances, such as one or more solubilizing agents, such as polysorbate 20, polysorbate 40, polysorbate 60 or polysorbate 80. A composition of the present invention may also contain a dispersant, such as lecithin or glycerine. Collagen may also be added.

Embodiments of compositions of the present invention may also comprise cyclodextrins, vitamin E, particularly in a solubilized form, and other antioxidants. Furthermore, compositions of the present invention may also comprise other ingredients, including sodium carbonate (from about 0.1% to about 5.0%), potassium chloride (from about 0.01% to about 1.0%), sodium citrate (from about 0.01% to about 5.0%), sodium thiosulfate (from about 0.01% to about 5.0%), sodium bisulfite, acetic acid, dextrose, magnesium chloride, alginic acid, and sodium borate.

Yet further embodiments of compositions of the present invention may further comprise a steroid, including but not limited to: hydrocortisone (from about 0.1% to about 10%), prednisone (from about 0.01% to about 10%), fluorometholone acetate (from about 0.01% to about 10%), dexamethasone sodium phosphate (from about 0.001% to about 1%), dexamethasone (from about 0.001% to about 1%), suprofen (from about 0.1% to about 10%), fluorometholone (from about 0.001% to about 1%), and medrysone (from about 0.1% to about 10%).

Still further compositions of the present invention may comprise, without limitation, betaxolol hydrochloride (from about 0.1% to about 10%), cyclopentolate hydrochloride (from about 0.1% to about 10%), p-phenylephrine hydrochloride (from about 0.1% to about 30%), epinephrine (from about 0.01% to about 20%), apraclonidine hydrochloride (from about 0.1% to about 10%), atropine sulfate (from about 0.1% to about 5%), carbachol (from about 0.1% to about 5%), pilocarpine hydrochloride (about 0.25%, 0.5%, 1%, 2%, 3%, 4%, 5%, 6%, 8%, and 10%), sulfacetamide sodium (from about 0.1% to about 30%), homatropine hydrobromide (from about 0.5% to about 10%), scopolamine hydrobromide (from about 0.1% to about 5%), tropicamide (from about 0.1% to about 5%), naphazolinehydrochloride (from about 0.01% to about 5%), tetrahydrozoline hydrochloride (from about 0.001% to about 5%), oxymetazoline hydrochloride (from about 0.001% to about 5%), ketorolac tromethamine (from about 0.001% to about 5%), levobunolol hydrochloride (from about 0.001% to about 5%), idoxuridine (from about 0.01% to about 2%), trimethoprim (from about 0.1 to about 5.0 mg/gm), dipivefrin hydrochloride (from about 0.01% to about 5%,), metipranolol (from about 0.01% to about 5%), trifluridine (from about 0.01% to about 5%), diclofenac sodium (from about 0.01% to about 5%), zinc isoflurophate (from about 0.01% to about 3%), demecarium bromide (from about 0.01% to about 5%), timolol maleate (from about 0.01% to about 5%), carteolol hydrochloride (from about 0.5 to about 25.0 mg/gm), and vidrabine (from about 0.1% to about 15%).

The ophthalmic gels of the present invention provide novel combination of compounds in a viscous formulation with benefits that heretofore were not obvious. The potential benefits include better sterility during ocular surface preparation, improved anesthesia and patient comfort, and an overall easier and quicker procedure.

Prior to the present invention, conventional methods for sterilization of the ocular surface prior to any intraocular procedure required multiple applications of antiseptic to the eye. First, the eyelids were wiped one or more times with antiseptic soaked gauze. Then the eyelashes were swabbed with a cotton-tip applicator soaked in antiseptic. Finally, the conjunctiva and ocular fornices were irrigated with an antiseptic. All of these maneuvers were intended to provide complete contact of the antiseptic with bacteria around the eye and render them harmless. Unfortunately, due to the contour of the eye and eyelids, most of the antiseptic was lost due to run-off; therefore, limiting the beneficial amount of contact time between the antiseptic and the bacteria.

Prior to the present invention, anesthesia for IVI involved using a topical anesthetic prior to sterile antiseptic preparation followed by additional topical anesthetic on a cotton swab and an injection of subconjunctival anesthetic at the intended IVI site. Theoretically, these steps added additional risks for re-introducing bacteria to the injection site in many ways. First of all, the additional anesthetic was given after the iodine prep. These simple steps also added multiple supplies to the procedure, with each item potentially increasing the risk of contamination. These additional supplies included a filtered and non-filtered needle, syringe, cotton swab, bottle of topical anesthetic, and a vial of injectible anesthetic. The technical maneuver of a subconjuctival injection often required awkward positioning and risk needle contact with potentially unsterile lids and lashes, which was then introduced into the subconjuctival space. The compositions of the present invention eliminate multiple steps and supplies and potentially result in a lower risk of infection.

The present invention comprises a one step method for placing a composition taught herein onto the eye, instead of the more labor intensive, multi-step method described above for providing a sterile field and anesthesia. Not only do the gel compositions of the present invention thoroughly coat the ocular structures, but they also produce improved contact time and bacterial inhibition, compared with conventional compositions and methods. In addition, gel composition application according to the present invention limits additional bacteria that might be liberated from eyelid glands during the manipulation using conventional surface preparation materials and methods. Using the compositions and methods of the present invention, all of these benefits are achieved in substantially less time than that previously required to prepare the surface of an eye using conventional methods and materials.

Beyond the examples given above in the field of ophthalmology, the attributes of compositions and methods of the present invention also improve medical treatment, surgical interventions, and wound care in other medical disciplines. As an additional method of the use of compositions according to the present invention, a gel or semi-gel composition comprising antiseptic agents and anesthetic agents may be applied to the tympanic membrane prior to myringotomy procedures. In such an example, the tympanic membrane is an anatomic surface which must be incised as part of the planned treatment procedure, and where both antisepsis and anesthesia are desirable prior to such treatment. Because of its adherent properties, a gel or semi-gel composition including antiseptic agents and anesthetic agents provides better surface adhesion and topical activity than liquid applications of similar agent components. Moreover, an aqueous gel or semi-gel composition of the present invention is more easily removed by irrigation when desired from the tympanic membrane surface than would be the case with a non-aqueous gel or ointment composition.

As yet another method of the use of compositions of the present invention, a gel or semi-gel composition comprising antiseptic agents and anesthetic agents may be applied to a laceration, abrasion, burn, or other cutaneous wound prior to treatment. Topical anesthetics such as lidocaine would normally have too little absorbance through the entire dermis to permit use for dermal anesthesia. However, with an open wound, direct topical activity of the antiseptic and anesthetic agents of a gel or semi-gel composition of the present invention is sufficient to allow for effective treatment therein.

In still other methods of the use of compositions of the present invention, a gel or semi-gel composition including antiseptic agents and anesthetic agents may be applied to anatomic surfaces such as mucous membranes in the mouth during dental and oral surgical procedures for one-step application of antiseptic and anesthetic treatment. Such dental and oral surgical procedures may include dental fillings, endodontic, orthodontic, periodontal, or dental implant treatments and surgery.

Compositions of the present invention for treatment of an anatomic surface comprise an effective amount of an antiseptic agent, an effective amount of an anesthetic agent, and an aqueous gel or semi-gel formulation base. Such compositions of the present invention for treatment of an anatomic surface may be administered to a patient in preparation for a treatment or invasive procedure. An antiseptic agent in compositions of the present invention for treatment of an anatomic surface may comprise povidone-iodine. An anesthetic agent in compositions of the present invention for treatment of an anatomic surface may comprise lidocaine, tetracaine, proparacaine, or bupivacaine. An aqueous gel or semi-gel formulation base in compositions of the present invention for treatment of an anatomic surface may comprise a cellulose derivative. An aqueous gel or semi-gel formulation base in compositions of the present invention for treatment of an anatomic surface may comprise a methyl cellulose or carboxymethyl cellulose salt. An aqueous gel or semi-gel formulation base in compositions of the present invention for treatment of an anatomic surface may comprise aqueous cross-linked acrylic polymers, poly acrylic acid, pluronic polyol polymers, other polyols, carboxy vinyl polymers, or other carbomers. An aqueous gel or semi-gel formulation base in compositions of the present invention for treatment of an anatomic surface may comprise an aqueous gel or semi-gel at a desired viscosity in the range of about 10,000 cps to about 50,000 cps at about 25° C. An aqueous gel or semi-gel formulation base in compositions of the present invention for treatment of an anatomic surface may comprise hydroxypropyl cellulose, methyl hydroxypropyl cellulose, hydroxypropyl methyl cellulose, cellulose acetate, ethyl cellulose, methyl hydroxyethyl cellulose, hydroxyethyl cellulose, cellulose gum, dextran, polyvinyl alcohol, poyvinylacyrlates, or polymeric mixtures thereof. Compositions of the present invention for treatment of an anatomic surface may comprise a steroid. Compositions of the present invention for treatment of an anatomic surface may comprise an antibiotic.

Methods for a treatment of an anatomic surface of the present invention comprise a). contacting an anatomic surface with a composition comprising an effective amount of an antiseptic agent, an effective amount of an anesthetic agent, and a gel or semi-gel formulation base; and, b). allowing said composition to remain in contact with said surface for a desired period of time to achieve desired antiseptic and anesthetic treatment of said surface. An anatomic surface for treatment using methods of the present invention may comprise a cornea, a tympanic membrane, a mucous membrane, or a wound, such as a laceration, burn, or abrasion.

A method of the present invention of antisepsis and anesthesia for an ocular surface comprises: a) applying in one-step a composition comprising a gel or semi-gel formulation base, an anesthetic agent, and an antiseptic agent to said surface, and b) allowing said composition to remain in contact with said ocular surface for a desired period of time.

Although the foregoing embodiments of the present invention have been described in some detail by way of illustration and example for purposes of clarity and understanding, it will be apparent to those skilled in the art that certain changes and modifications may be practiced within the spirit and scope of the present invention. Therefore, the descriptions presented herein should not be construed to limit the scope of the present invention, the essential features of which are set forth in the appended claims.

What is claimed is:

1. A composition for treating an anatomic surface, comprising an effective amount of an antiseptic agent, an effective amount of an anesthetic agent, and an aqueous gel or semi-gel formulation base.

2. The composition of claim 1, wherein said composition is administered to a patient in preparation for a treatment or invasive procedure.

3. The composition of claim 1, wherein said antiseptic agent comprises povidone-iodine.

4. The composition of claim 1, wherein said anesthetic agent comprises lidocaine, tetracaine, proparacaine, or bupivacaine.

5. The composition of claim 1, wherein said aqueous gel or semi-gel formulation base is a cellulose derivative.

6. The composition of claim 1, wherein said aqueous gel or semi-gel formulation base is a methyl cellulose or carboxymethyl cellulose salt

7. The composition of claim 1, wherein said aqueous gel or semi-gel formulation base is an aqueous cross-linked acrylic polymers, poly acrylic acid, pluronic polyol polymers, other polyols, carboxy vinyl polymers, or other carbomers.

8. The composition of claim 1, wherein said aqueous gel or semi-gel formulation base forms an aqueous gel or semi-gel at a desired viscosity in the range of about 10,000 cps to about 50,000 cps at about 25° C.

9. The composition of claim 1, wherein said aqueous gel or semi-gel formulation base is hydroxypropyl cellulose, methyl hydroxypropyl cellulose, hydroxypropyl methyl cellulose, cellulose acetate, ethyl cellulose, methyl hydroxyethyl cellulose, hydroxyethyl cellulose, cellulose gum, dextran, polyvinyl alcohol, poyvinylacrylates, or polymeric mixtures thereof.

10. The composition of claim 1, further comprising a steroid.

11. The composition of claim 1, further comprising an antibiotic.

12. A method for treatment of an anatomic surface comprising:
a) providing a composition comprising an effective amount of an antiseptic agent, an effective amount of an anesthetic agent, and a gel or semi-gel formulation base to an anatomic surface; and
b) contacting said composition with said surface for a desired period of time to achieve desired antiseptic and anesthetic treatment of said surface.

13. The composition of claim 12, wherein said anatomic surface is a cornea.

14. The composition of claim 12, wherein said anatomic surface is a tympanic membrane.

15. The composition of claim 12, wherein said anatomic surface is a mucous membrane.

16. The composition of claim 12, wherein said anatomic surface comprises a wound.

17. The composition of claim 16, wherein said wound is a laceration.

18. The composition of claim 16, wherein said wound is an abrasion.

19. The composition of claim 16, wherein said wound is a burn.

20. A one-step method of antisepsis and anesthesia for an ocular surface comprising: a) providing a composition comprising a gel or semi-gel formulation base, an anesthetic agent, and an antiseptic agent to an ocular surface, and b) contacting said composition and said ocular surface for a desired period of time.
